(19) United States
(12) Patent Application Publication
(10) Pub. No.: US 2019/0167208 A1
(43) Pub. Date: Jun. 6, 2019

(54) BIO-INFORMATION PROCESSING APPARATUS AND BIO-INFORMATION PROCESSING METHOD

(71) Applicant: SAMSUNG ELECTRONICS CO., LTD., Suwon-si (KR)

(72) Inventors: Yeol Ho LEE, Anyang-si (KR); Kak NAMKOONG, Seoul (KR); Myoung Hoon JUNG, Bucheon-si (KR); Won Jong JUNG, Seoul (KR)

(73) Assignee: SAMSUNG ELECTRONICS CO., LTD., Suwon-si (KR)

(21) Appl. No.: 16/206,087

(22) Filed: Nov. 30, 2018

(30) Foreign Application Priority Data
Dec. 1, 2017 (KR) 10-2017-0164558

(51) Int. Cl. A61B 5/00; A61B 5/11; A61B 5/1477

(52) U.S. Cl. CPC A61B 5/7278; A61B 5/4875; A61B 5/681; A61B 5/7475; A61B 5/1477; A61B 5/746; A61B 5/7275; A61B 5/1123; A61B 5/7425; A61B 5/486

(57) ABSTRACT

A bio-information processing apparatus may include: a main body; a strap which is connected to both ends of the main body; an impedance measurer configured to measure a bio-impedance of a user while the main body and the strap are in contact with the user; and a processor configured to estimate a body water amount of the user by applying the measured bio-impedance to a body water estimation model.

### CROSS-REFERENCE TO RELATED APPLICATION(S)

This application claims priority from Korean Patent Application No. 10-2017-0164558, filed on Dec. 1, 2017 in the Korean Intellectual Property Office, the disclosure of which is incorporated herein by reference in its entirety.

### BACKGROUND

### 1. Field

Apparatuses and methods consistent with exemplary embodiments relate to processing bio-information, and more particularly to processing bio-information obtained from an object.

### 2. Description of the Related Art

Measurement of body composition using Bio Impedance Analysis or Bioelectrical Impedance Analysis (BIA) may be performed by using a whole-body apparatus. The Bio Impedance Analysis using the whole-body apparatus is generally performed by analyzing voltage detected after applying a current to electrodes disposed at the position of hands and feet.

However, the Bio Impedance Analysis using the whole-body apparatus has restrictions on movement and installation due to the size of the whole-body apparatus and supply of power, thereby limiting time and space of the analysis of body composition.

That is, body composition may not be measured on the spot and at any time desired by a user, such that a user doing an outdoor activity or an emergency patient may not be handled at a proper time, and there is difficulty in determining prognosis after treatment.

### SUMMARY

Exemplary embodiments address at least the above problems and/or disadvantages and other disadvantages not described above. Also, the exemplary embodiments are not required to overcome the disadvantages described above, and may not overcome any of the problems described above.

According to an aspect of an exemplary embodiment, there is provided a bio-information processing apparatus, including: a main body; a strap which is connected to both ends of the main body; an impedance measurer configured to measure a bio-impedance of a user while the main body and the strap are in contact with the user; and a processor configured to estimate a body water amount of the user by applying the measured bio-impedance to a body water estimation model.

The processor may convert the measured bio-impedance into at least one of a linear expression, a fractional expression, and an exponential expression, and may estimate the body water amount of the user by applying the converted bio-impedance to the body water estimation model.

The bio-information processing apparatus may further include an input interface configured to input at least one of user information and a usage mode, and the user information may include at least one of a gender of the user, an age of the user, a stature of the user, a weight of the user, a body mass index (BMI) of the user, and a measurement position of the user.

The processor may apply a weighted value to at least one of the user information and the measured bio-impedance, and may estimate the body water amount by applying a result, which is obtained by applying the weighted value to the at least one of the user information and the measured bio-impedance, to the body water estimation model.

The usage mode may include a normal mode, an exercise mode, and a patient mode.

In response to the usage mode being the normal mode, the processor may generate water intake guidance information based on the input user information and the estimated body water amount.

In response to the usage mode being the exercise mode, the processor may continuously estimate a change in the estimated body water amount, and in response to the change in the estimated body water amount exceeding a predetermined threshold value, the processor may generate warning information.

In response to the usage mode being the patient mode, the processor may generate at least one of a prediction index of diseases, which include liver cirrhosis, intercapillary glomerulosclerosis, and edema, and prognosis evaluation information of a surgical operation field based on the input user information and the estimated body water amount.

The processor may determine whether the user takes a break by monitoring movement of the user, and may generate an alarm to induce measurement of the body water amount in response to determining that the user takes a break.

The impedance measurer may include: a first rear surface electrode and a second rear surface electrode which are disposed on a rear surface of the main body to directly come into contact with the user; and a first front surface electrode and a second front surface electrode which are disposed on a surface of the main body to come into contact with the user when body composition of the user is measured, and which are arranged to be touchable by a single finger of the user.

The impedance measurer may apply a current through the first rear surface electrode and the first front surface electrode, and may measure the bio-impedance by measuring voltage between the second rear surface electrode and the second front surface electrode.

Each of the first and second front surface electrodes and the rear first and second surface electrodes may be formed in at least one shape of a square, a circle, a concentric circle, and a semi-circle.

The bio-information processing apparatus may further include an output interface configured to output at least one of a body water amount, a daily water intake amount, a recommended fluid intake amount, water intake guidance information, warning information, and a prediction index of diseases.

According to an aspect of another exemplary embodiment, there is provided a bio-information processing method including: measuring a bio-impedance; and converting the measured bio-impedance into at least one of a linear expression, a fractional expression, and an exponential expression, and estimating a body water amount of a user by applying the converted bio-impedance to a body water estimation model.

The bio-information processing method may further include inputting at least one of user information and a usage mode, wherein the user information may include at least one of a gender of the user, an age of the user, a stature of the user, a weight of the user, a body mass index (BMI) of the user, and a measurement position of the user.

The estimating the body water amount may include: applying a weighted value to at least one of the user information and the measured bio-impedance; and estimating the body water amount by applying a result, which is obtained by applying the weighted value to the at least one of the user information and the measured bio-impedance, to the body water estimation model.

In response to the usage mode being a normal mode, the bio-information processing method may further include generating water intake guidance information based on the user information and the estimated body water amount.

The bio-information processing method may further include, in response to the usage mode being an exercise mode, continuously estimating a change in the estimated body water amount, and in response to the change in the estimated body water amount exceeding a predetermined threshold value, generating warning information.

In response to the usage mode being a patient mode, the bio-information processing method may further include generating at least one of a prediction index of diseases, which include liver cirrhosis, intercapillary glomerulosclerosis, and edema, and prognosis evaluation information of a surgical operation field based on the user information and the estimated body water amount.

The bio-information processing method may further include outputting at least one of a body water amount, a daily water intake amount, a recommended fluid intake amount, water intake guidance information, warning information, and a prediction index of diseases.

### BRIEF DESCRIPTION OF THE DRAWINGS

The above and/or other aspects will be more apparent by describing certain exemplary embodiments, with reference to the accompanying drawings, in which:

FIG. 1 is a block diagram illustrating a bio-information processing apparatus according to an exemplary embodiment.

FIG. 2 is a block diagram illustrating a bio-information processing apparatus according to another exemplary embodiment.

FIGS. 3A, 3B, 3C, 3D, and 3E illustrate bio-information processing apparatuses according to various exemplary embodiments.

FIG. 4 is a flowchart illustrating a bio-information processing method according to an exemplary embodiment.

### DETAILED DESCRIPTION

Exemplary embodiments are described in greater detail below with reference to the accompanying drawings.

In the following description, like drawing reference numerals are used for like elements, even in different drawings. The matters defined in the description, such as detailed construction and elements, are provided to assist in a comprehensive understanding of the exemplary embodiments. However, it is apparent that the exemplary embodiments can be practiced without those specifically defined matters. Also, well-known functions or constructions are not described in detail since they would obscure the description with unnecessary detail.

Process steps described herein may be performed differently from a specified order, unless a specified order is clearly stated in the context of the disclosure. That is, each step may be performed in a specified order, at substantially the same time, or in a reverse order.

Further, the terms used throughout this specification are defined in consideration of the functions according to exemplary embodiments, and can be varied according to a purpose of a user or manager, or precedent and so on. Therefore, definitions of the terms should be made on the basis of the overall context.

Any references to singular may include plural unless expressly stated otherwise. In the present specification, it should be understood that the terms, such as ‘including’ or ‘having,’ etc., are intended to indicate the existence of the features, numbers, steps, actions, components, parts, or combinations thereof disclosed in the specification, and are not intended to preclude the possibility that one or more other features, numbers, steps, actions, components, parts, or combinations thereof may exist or may be added.

Expressions such as “at least one of,” when preceding a list of elements, modify the entire list of elements and do not modify the individual elements of the list. For example, the expression, “at least one of a, b, and c,” should be understood as including only a, only b, only c, both a and b, both a and c, both b and c, or all of a, b, and c.---_{—}

Hereinafter, exemplary embodiments of a bio-information processing apparatus and bio-information processing method will be described below with reference to the accompanying drawings.

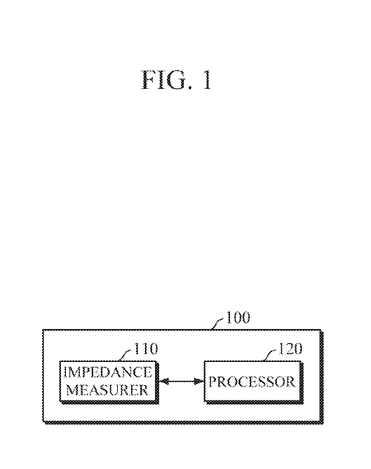

FIG. 1 is a block diagram illustrating a bio-information processing apparatus according to an exemplary embodiment.

Referring to FIG. 1, the bio-information processing apparatus 100 may measure bio-impedance of an object, and may estimate a body water amount based on the measured bio-impedance. The bio-information processing apparatus 100 may continuously measure the body water amount in a non-invasive manner.

Here, the body water amount may include at least one of Total Body Water (TBW), Intra Cellular Water (ICW), and Extra Cellular Water (ECW).

The bio-information processing apparatus 100 may be implemented as a wearable device and/or a mobile electronic device, such that the bio-information processing apparatus 100 may overcome restrictions on time and space, and may measure and provide bio-information even in various living environments of users.

For example, the bio-information processing apparatus 100 may continuously or periodically measure bio-impedance from ordinary users, athletes, or patients, and may provide health information for individual users by estimating a body water amount based on the measured impedance.

Hereinafter, description will be made based on an exemplary embodiment where the bio-information processing apparatus 100 is implemented as a wearable device which may be worn on the wrist, and may estimate a body water amount. However, the bio-information processing apparatus 100 is not limited thereto, and may estimate body fat and the percentage (%) of body fat base on the measured bio-impedance. The bio-information processing apparatus 100 may be implemented as a cellular phone, a smartphone, a tablet PC, a laptop computer, a personal digital assistant (PDA), a portable multimedia player (PMP), a navigation, an MP3 player, a digital camera, and various wearable devices.

Referring back to FIG. 1, the bio-information processing apparatus 100 includes an impedance measurer 110 and a processor 120.

The impedance measurer 110 may measure bio-impedance Z. The impedance measurer 110 may be also referred to as a body fat analyzer or an impedance measuring circuit.

For example, the impedance measurer 110 may include one or more electrodes which may contact an object to measure bio-impedance. For example, the impedance measurer 110 may include current electrodes and voltage electrodes. While the current and voltage electrodes are in contact with the object, the impedance measurer 110 may apply an alternating current to the current electrodes, may measure voltages from the voltage electrodes which are respectively paired with the current electrodes, and may determine the bio-impedance based on the applied current and the measured voltages. The impedance measurer 110 may use a two electrode method or a four electrode method.

Further, the impedance measurer 110 may include one or more electrodes which are provided in a combination of various sizes and shapes, and the distance between the electrodes may be adjusted according to features of a device implemented as the bio-information processing apparatus 100.

For example, in the case where the bio-information processing apparatus 100 is implemented as a wearable device which may be worn on the wrist, the impedance measurer 110 may measure bio-impedance by using a four-electrode method, with a pair of electrodes being disposed on a front surface of a main body of the device, and another pair of electrodes being disposed on a rear surface of the main body of the device.

In another example, the impedance measurer 110 may measure bio-impedance by using a four-electrode method, with a pair of electrodes being disposed on a rear surface of a main body of the wearable device which may be worn on the wrist, and another pair of electrodes being disposed on one side (particularly on a surface contacting the object) of a strap which fastens the main body of the device to the object. In this case, the main body of the wearable device and the strap may be electrically connected with each other to apply a current to the electrodes disposed on the strap.

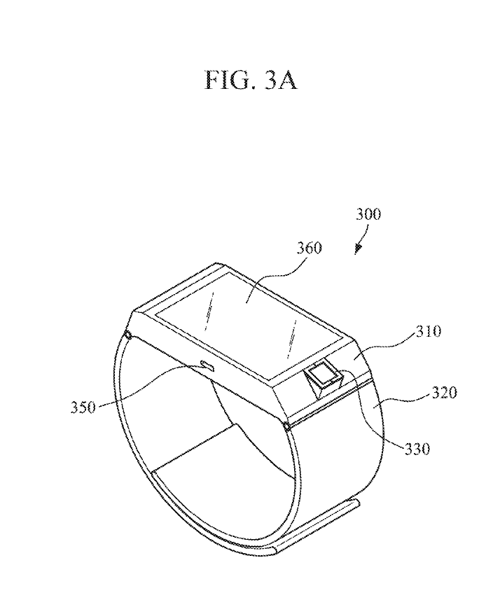

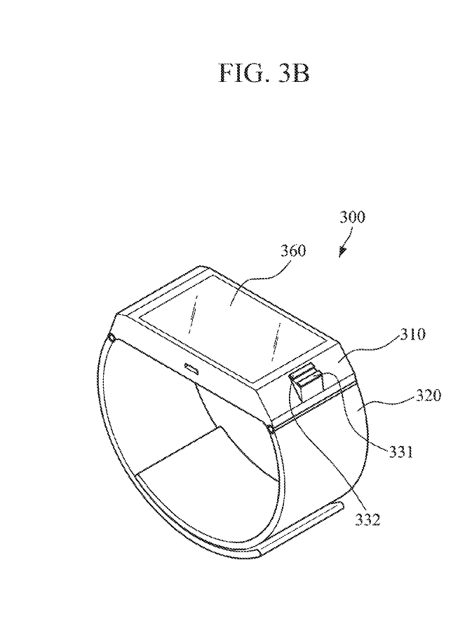

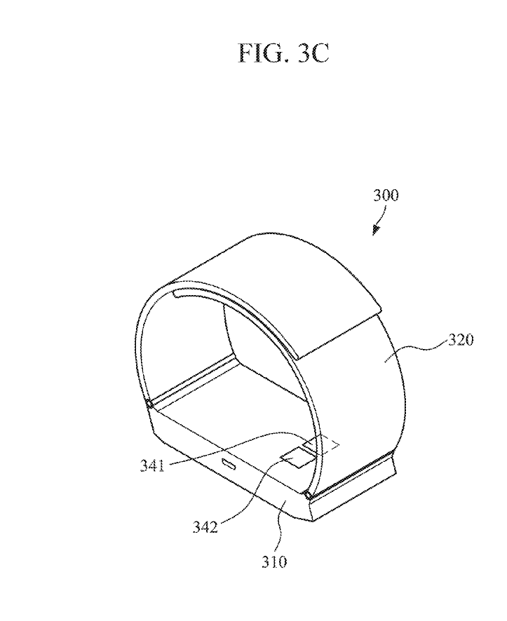

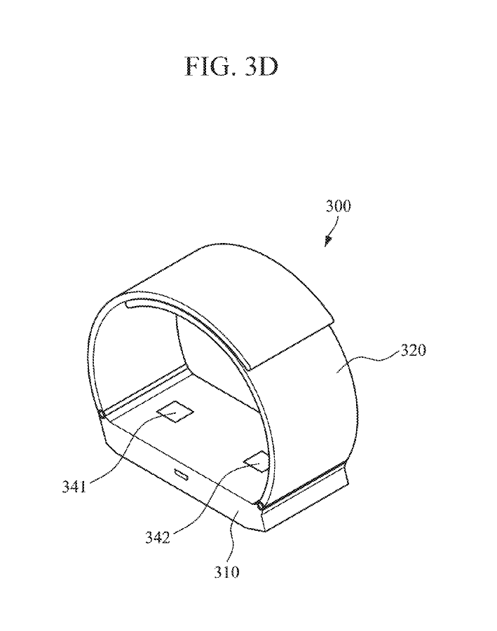

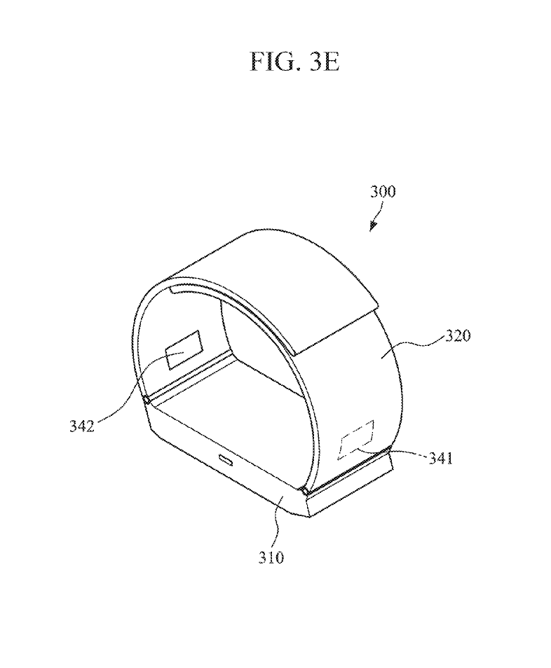

However, the impedance measurer 110 is not limited thereto, and may include electrodes in various shapes and arrangements, which will be described later in detail with reference to FIGS. 3A to 3E.

The processor 120 may estimate a body water amount of a user by applying the measured bio-impedance to a body water estimation model.

Here, the body water estimation model may be a regression analysis model, which is pre-generated based on a correlation between a body water amount, measured by an apparatus for measuring a reference body water amount, and bio-impedance, or may be a learning model generated by machine learning. In addition, the body water estimation model may be estimation models which are classified into one or more groups according to at least one criterion among a user's gender, age, stature, weight, body mass index (BMI), and measurement position.

For example, the processor 120 may select a body water estimation model according to user information, a measurement position, and a usage mode, and may estimate a body water amount based on the selected body water estimation model.

For example, the processor 120 may convert the measured bio-impedance into the form of at least one of a linear expression, a fractional expression, and an exponential expression, and may estimate the body water amount of a user by applying the converted bio-impedance to the selected body water estimation model.

Once the bio-impedance of a user is measured by the impedance measurer 110, the processor 120 may convert the measured bio-impedance Z into the form of a monomial expression, a fractional expression, an exponential expression, and a linear expression (e.g., α*Z, α/Z, α*Z β, etc.), in which α and β may denote predetermined constant values or user information. For example, the processor 120 may convert the measured bio-impedance Z of the user based on the user's stature. Specifically, the processor 120 may obtain the user's stature from the user information, raise the user's stature to the power of n to obtain the value of statureⁿ, and divide the value of statureⁿ by the measured bio-impedance Z to obtain the value of statureⁿ/Z. For example, n may denote a positive integer. The processor 120 may apply the value of statureⁿ/Z to the body water estimation model. However, the operation of the processor 120 is not limited thereto, and in the case where the constant applied when converting the bio-impedance is 1, the processor 120 may omit the process of converting the measured bio-impedance Z, and may apply the measured bio-impedance to the body water estimation model.

In another example, the processor 120 may estimate a body water amount by applying user information, which includes at least one of gender, age, stature, weight, BMI, and measurement position of a user, and the measured bio-impedance Z to the body water estimation model.

For example, the following Equation 1 may be an example of the body water estimation model.

Body water amount estimated=a0+a1×gender+a2×age+a3×stature+a4×weight+a5×impedance (Z) [Equation 1]

Herein, a0, a1, a2, a3, a4, and a5 may be constant values or functions. For example, a0 may be an offset parameter, and a0, a1, a2, a3, a4, and a5 may be constant values predetermined when the body water estimation model is generated based on a correlation between a body water amount and a user's gender, age, stature, weight, BMI, measurement position, and/or the bio-impedance Z. For example, the processor 120 may determine the offset parameter a0 according to a change in a measurement position of the bio-impedance, and may determine the values a1, a2, a3, a4, and a5 by considering an effect of the user information on the estimation of the body water amount.

Based on the types of the input user information, and whether the user information is input, the processor 120 may apply the user information to the body water estimation model, thereby optimizing the use of a limited calculation environment. For example, in the case where ‘gender’ is not input, the processor 120 may define a value of ‘gender’ as 0, and may exclude the calculation performed based on ‘gender’ from the body water estimation model.

Further, the processor 120 may apply a weighted value to at least one of the user information and the measured bio-impedance, and may estimate the body water amount by applying a result, obtained by applying the weighted value, to the body water estimation model.

For example, the body water amount is generally proportional to the stature and weight, and is inversely proportional to age, and men usually have a higher percentage of body water than women. Accordingly, the processor 120 may apply a weighted value to at least one of the user information and the measured bio-impedance, and may estimate an individualized body water amount by applying a result, obtained by applying the weighted value, to the body water estimation model.

The processor 120 may generate guidance information or warning information based on a measurement result of the body water amount according to at least one usage mode among a normal mode, an exercise mode, a patient mode, and a user-defined mode.

Here, the normal mode may be an initial setting mode of the bio-information processing apparatus 100, and may be maintained unless a user changes the usage mode.

Further, the exercise mode may be selected by a user who is an athlete, a user being on a diet, or a user having a large amount of muscles for their weight. In the case where a usage mode is changed to the exercise mode, the processor 120 may calculate a change in the body water amount by continuously or periodically estimating body water.

In the patient mode, indices of various diseases may be evaluated by using intracellular water and extracellular water. In the case where a usage mode is changed to the patient mode, the processor 120 may estimate the intracellular water and the extracellular water to track a change therein.

The user-defined mode is a usage mode defined based on at least one of a purpose of use of the bio-information processing apparatus 100, the input user information, a measurement position, and a measurement period of impedance. For example, if the input user information includes an age of ‘thirties’, a gender of ‘male’, a weight of ‘70 kg’, and a measurement position of ‘wrist’, the processor 120 may select a body water estimation model, which corresponds to the input user information, from among one or more body water estimation models. In this case, the bio-information processing apparatus 100 may allow a user to set the bio-impedance measurement period to be a period of once per hour (once/hour) based on an environment where the bio-information processing apparatus 100 is used.

However, the user-defined mode is not limited thereto, and if the user-defined mode is set to an ‘automatic setup’, the processor 120 may detect whether a user takes a break or does an activity based on a motion of the bio-information processing apparatus 100, which is sensed by a motion sensor, and the processor 120 may automatically change the usage mode according to a user's body type (e.g., thin, normal, mildly obese, obese, muscular, etc.) based on the amount of body water, body fat, and muscle, which is estimated by using the measured bio-impedance. For example, with weight being equal, a person having a large amount of muscle may have a higher percentage of body water, such that the processor 120 may change the usage mode to the ‘exercise mode’ based on the estimated body water amount of the user.

For example, in the case where the usage mode is the normal mode, the processor 120 may generate water intake guidance information based on the input user information and the estimated body water amount. For example, the processor 120 may compare a reference body water amount with the estimated body water amount, and if the body water amount of a user is less than the reference body water amount by a predetermined amount (e.g., 2% less than the reference body water amount), the processor 120 may generate body water intake guidance information to induce a user to intake water.

Here, the reference body water amount may be an average body water amount according to a user's gender, age, and weight, and may be received from an external device, or may be a body water amount which is initially estimated at the initialization of the bio-information processing apparatus 100 and/or according to a user's request.

In another example, in the case where the usage mode is the exercise mode, the processor 120 may continuously estimate a change in the estimated body water amount, and in the case where the change in the estimated body water amount exceeds a predetermined threshold value, the processor 120 may generate warning information.

For example, while a user does an exercise or an activity which requires physically intense movements, loss of body water may result in a reduced exercise performance. In this case, if sufficient water is not supplied such that a pituitary gland may not maintain homeostasis of body water, problems may occur in regulation of body temperature or in the cardiovascular system.

Accordingly, if the usage mode is the exercise mode, the processor 120 may measure bio-impedance of a user according to a predetermined period (e.g., ten times/hour), and may estimate a body water amount based on the measured bio-impedance, in which the processor 120 may continuously estimate a change between the initially estimated body water amount or a body water amount estimated immediately before the current estimation, and the currently estimated body water amount.

In this case, if the currently estimated body water amount is less than the initially estimated body water amount by a predetermined amount (e.g., 2% less than the initially estimated body water amount), or in the case where the currently estimated body water amount is less than the body water amount estimated immediately before the current estimation by a predetermined amount (e.g., 0.4% less than the body water amount estimated immediately before the current estimation), the processor 120 may generate warning information to warn of a sharp decrease in the body water amount. In this case, the warning information may include one of an amount of body water to be supplemented, an exercise duration time, and a warning to stop exercise.

As described above, the processor 120 may continuously estimate the body water amount based on the bio-impedance, and may calculate a change in the body water amount, such that even in the case where the body water amount is changed during a short period of time due to an intense activity such as an exercise, the processor 120 may generate warning information to maintain exercise performance of a user.

In yet another example, if the usage mode is the patient mode, the processor 120 may generate a prediction index of diseases including liver cirrhosis, intercapillary glomerulosclerosis, and edema, and prognosis evaluation information of a surgical operation field based on the input user information and the estimated body water amount.

For example, the human body water may be divided into intracellular water and extracellular water, a ratio of which is maintained at 3:2. In this case, the processor 120 may calculate a prediction index of diseases, including liver cirrhosis, intercapillary glomerulosclerosis, and edema, according to an extent that the estimated ratio of the intracellular water and the extracellular water falls outside a predetermined reference ratio (e.g., intracellular water:extracellular water=3:2).

Based on the calculated prediction index of diseases and prognosis evaluation information of the surgical operation field, the processor 120 may generate guidance information regarding water intake or change of a resting posture, or may transmit an alarm (e.g., text message, warning information, etc.) to a related institution (e.g., specialized hospital, sanatorium, nursing care organization, etc.).

Further, the processor 120 may generate an alarm to periodically, constantly, or continuously measure the body water amount based on a user movement state and/or a measurement state of impedance which is measured by the impedance measurer 110.

For example, in the case where the bio-information processing apparatus 100 is implemented as a wearable device which may be worn on the wrist, impedance may not be stably measured by the impedance measurer 110 due to contact failure or motion noise caused while a user moves.

For example, the processor 120 may detect whether a user takes a break or does an activity based on the motion of the bio-information processing apparatus 100 which is sensed by a motion sensor (e.g., acceleration sensor, gyro sensor, proximity sensor, image sensor, etc.), and may generate an alarm to measure the body water amount by determining a current activity state of a user.

For example, in the case where a user movement is detected by the motion sensor, the bio-impedance Z measured by the impedance measurer 110 may include many motion noises, or the measured impedance may be inaccurate due to incomplete contact of electrodes. Accordingly, the processor 120 monitors a user movement by using the motion sensor, and in the case where a user movement is not detected, the processor 120 determines whether a user takes a break. Upon determination, if the user takes a break or in a stable condition, the processor 120 may generate an alarm to induce measurement of the body water amount. In this manner, the processor 120 may accurately measure the body water amount by inducing measurement of the body water amount while a user in a stable state, and may constantly manage the body water amount.

However, even in the case where a user movement is detected by the motion sensor, if a voltage, detected on two or four electrodes of the impedance measurer 110, is maintained in a normal range, or a current and a voltage are constantly detected on the two or four electrodes, such that the processor 120 determines that the electrodes normally contact an object, the processor 120 may constantly estimate the body water amount by continuously measuring bio-impedance through the impedance measurer 110.

In addition, in the case where the bio-information processing apparatus 100, as a mobile device, is a wearable device which may be worn on the wrist, a motion caused in a user's daily life may constantly occur. In this case, the processor 120 may monitor motion information regardless of a usage mode (e.g., normal mode, exercise mode, patient mode, user-defined mode, etc.), and may generate a notification to induce measurement of the body water amount at a time when the motion is stopped, or may automatically measure a user's body water amount.

In this manner, by measuring the body water amount based on a user movement state and/or a measurement state of impedance measured by the impedance measurer 110, the processor 120 may continuously/periodically measure the body water amount even in constant motion.

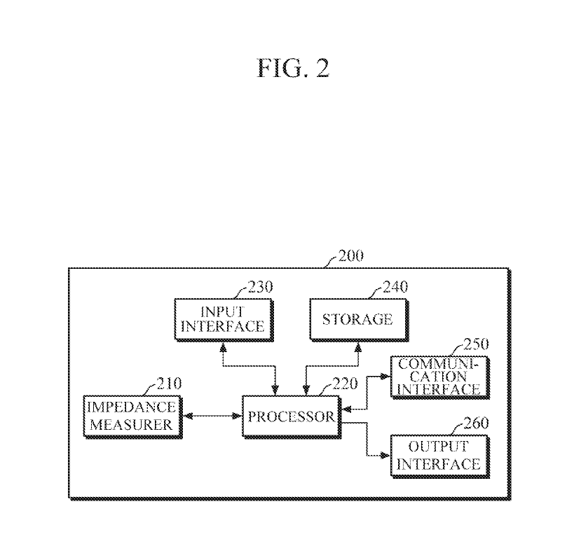

FIG. 2 is a block diagram illustrating another example of a bio-information processing apparatus.

Referring to FIG. 2, the bio-information processing apparatus 200 includes an impedance measurer 210, a processor 220, an input interface 230, a storage (e.g., a memory) 240, a communication interface 250, and an output interface 260. Here, the impedance measurer 210 and the processor 220 basically perform the same function as the impedance measurer 110 and the processor 120 described above with reference to FIG. 1, such that description below will be made based on details that do not overlap.

The input interface 230 may receive input of various operation signals and information required for estimation of body water from a user. For example, the input interface 230 may include a keypad, a dome switch, a touch pad (static pressure/capacitance), a jog wheel, a jog switch, a hardware (H/W) button, and the like. Particularly, the touch pad, which forms a layer structure with a display, may be called a touch screen.

For example, the input interface 230 may receive input of the user information, which includes at least one or more of gender, age, stature, weight, BMI, and a measurement position of users, and a usage mode.

Once the user information is input through the input interface 230, the processor 220 may select a body water estimation model, which is generated by being classified into one or more groups, based on the input user information.

The storage 240 may store programs or commands for operation of the bio-information processing apparatus 200, and may store data input to and output from the bio-information processing apparatus 200. For example, the storage 240 may store the user information input through the input interface 230, the bio-impedance data measured by the impedance measurer 210, a body water estimation model, and the like.

The storage 240 may include at least one storage medium of a flash memory type memory, a hard disk type memory, a multimedia card micro type memory, a card type memory (e.g., an SD memory, an XD memory, etc.), a Random Access Memory (RAM), a Static Random Access Memory (SRAM), a Read Only Memory (ROM), an Electrically Erasable Programmable Read Only Memory (EEPROM), a Programmable Read Only Memory (PROM), a magnetic memory, a magnetic disk, and an optical disk, and the like. Further, the bio-information processing apparatus 200 may operate an external storage medium, such as web storage and the like, which performs a storage function of the storage 240 on the Internet.

The communicator 250 may perform communication with an external device. For example, the communicator 250 may transmit the user information input from a user through the input interface 230, the bio-impedance data measured by the impedance measurer 210, a body water estimation result of the processor 220, and the like to the external device, or may receive, from the external device, the user information, the bio-impedance of the user, the body water estimation model, the reference body water amount, and various data about many diseases according to a usage mode.

Further, the communicator 250 may transmit an alarm or warning information in the form of a text message or a warning signal based on the calculated prediction index of diseases and prognosis evaluation information of a surgical operation field to a related institution (e.g., specialized hospital, sanatorium, nursing care organization, etc.).

In this case, the external device may be medical equipment using a body water estimation model database (DB) and/or a body water estimation result, a printer to print out results, or a display to display the body water estimation result. In addition, the external device may be a digital TV, a desktop computer, a cellular phone, a smartphone, a tablet PC, a laptop computer, a personal digital assistant (PDA), a portable multimedia player (PMP), a navigation, an MP3 player, a digital camera, a wearable device, and the like, but is not limited thereto.

The communicator 250 may communicate with an external device by using Bluetooth communication, Bluetooth Low Energy (BLE) communication, Near Field Communication (NFC), WLAN communication, Zigbee communication, Infrared Data Association (IrDA) communication, Wi-Fi Direct (WFD) communication, Ultra-Wideband (UWB) communication, Ant+ communication, WIFI communication, Radio Frequency Identification (RFID) communication, 3G communication, 4G communication, 5G communication, and the like. However, this is merely exemplary and is not intended to be limiting.

The output interface 260 may output at least one or more of the body water estimation result and the warning information under the control of the processor 220.

For example, the output interface 260 may output at least one of a body water amount, a daily water intake amount, a recommended fluid intake amount, water intake guidance information, warning information, and the prediction index of diseases, by using at least one of an acoustic method, a visual method, and a tactile method. To this end, the output interface 260 may include a display, a speaker, a vibrator, and the like.

FIGS. 3A to 3E are diagrams illustrating yet another example of a bio-information processing apparatus.

Referring to FIG. 3A, a bio-information processing apparatus 300 may be an example of the bio-information processing apparatuses 100 and 200 implemented as a wearable device that may be worn on the wrist. The bio-information processing apparatus 300 includes a main body 310, which is worn on an object, and a strap 320 which is connected to both ends of the main body 310 and fastens the main body 310 to the object as the strap 320 is wrapped around the object with tension. Here, the strap 320 may be made of urethane, silicone, rubber, leather, and the like.

The main body 310 of the bio-information processing apparatus 300 may include modules related to measurement of bio-information, and modules related to additional functions (e.g., clock, alarm, music listening function or movie watching function, etc.). For example, as illustrated in FIG. 3, the main body 310 may include a sensor necessary for measurement of bio-information. In this case, an impedance measurer 330 for measuring bio-impedance may be mounted in the main body 310. Further, although not illustrated in FIG. 3, the main body 310 may include one or more processors or memories, or a processing module including a combination thereof.

A manipulator 350 for receiving various control commands of a user may be mounted in the main body 310. In this case, the manipulator 350 may include a power button function for inputting a command to turn on/off the power of the bio-information processing apparatus 300. Further, the main body 310 may include a display 360 to visually provide various types of information to a user, and may include a speaker, a haptic device, and the like which presents information to a user in a non-visual manner.

In addition, in the case where the display 360 includes a touchable display, the display 360 may perform the function of the manipulator 350 instead of or along with the manipulator 350, and may receive input of the user information, which includes at least one of gender, age, stature, weight, BMI of a user, and various information including a measurement position, and a usage mode, from the user.

FIGS. 3B to 3E are diagrams explaining the impedance measurer of the bio-information processing apparatus 300, in which FIG. 3B is a perspective view of the bio-information processing apparatus 300 according to an embodiment, and FIGS. 3C to 3E are perspective views illustrating the bio-information processing apparatus 300 of FIG. 3B upside down.

Referring to FIGS. 3B and 3C, the bio-information processing apparatus 300 includes a main body 310, a strap 320, a first front surface electrode 331, a second front surface electrode 332, a first rear surface electrode 341, and a second rear surface electrode 342.

For example, the first front surface electrode 331 and the second front surface electrode 332 are disposed on a surface of the main body 310 to directly come into contact with the body of an object. That is, the object may contact the first front surface electrode 331 and the second front surface electrode 332 with the hand wearing the bio-information processing apparatus 300 and a finger of the other hand.

Further, the first rear surface electrode 341 and the second rear surface electrode 342 are disposed on a rear surface of the main body 310 to directly come into contact with the body of the object. That is, the first rear surface electrode 341 and the second rear surface electrode 342 may be directly in contact with the wrist of the object.

In this case, while the first rear surface electrode 341 and the second rear surface electrode 342 are in contact with the wrist, a finger or a portion in the palm of the hand comes into contact with the first front surface electrode 331 and the second front surface electrode 332, such that the first front surface electrode 331 and the first rear surface electrode 341 form a closed circuit, a processor of the bio-information processing apparatus 300 applies a sinusoidal alternating current, and measures voltage between the second front surface electrode 332 and the second rear surface electrode 342, so as to calculate bio-impedance by using the applied current value and the measured voltage value.

Here, the first front surface electrode 331 and the second front surface electrode 332, and the first rear surface electrode 341 and the second rear surface electrode 342 may be in a square shape as illustrated in FIGS. 3B and 3C. However, the shape is not limited thereto, and may be other shapes including a square, a rectangle, a diamond, a concentric circle, a semi-circle, a shape of a facing strap, and the like, but there is no limitation on the shape.

In another example, the first rear surface electrode 341 and the second rear surface electrode 342 may be arranged in a direction perpendicular to the longitudinal direction of the strap 320 as illustrated in FIG. 3C, and may be arranged in the same direction as the longitudinal direction of the strap 320 as illustrated in FIG. 3D.

For example, the first rear surface electrode 341 and the second rear surface electrode 342 may be disposed on the inner side of the strap 320. In this case, the strap 320 may be electrically connected with the main body 310 to apply a current through the first rear surface electrode 341 and the second rear surface electrode 342.

Upon measuring the bio-impedance from the first front surface electrode 331 and the second front surface electrode 332, and the first rear surface electrode 341 and the second rear surface electrode 342, the processor of the bio-information processing apparatus 300 estimates a body water amount of a user by applying the measured bio-impedance to a body water estimation model, and may display at least one of the measured bio-impedance, the estimated body water amount, a daily water intake amount, a recommended fluid intake amount, water intake guidance information, warning information, and the prediction index of diseases on the display 360.

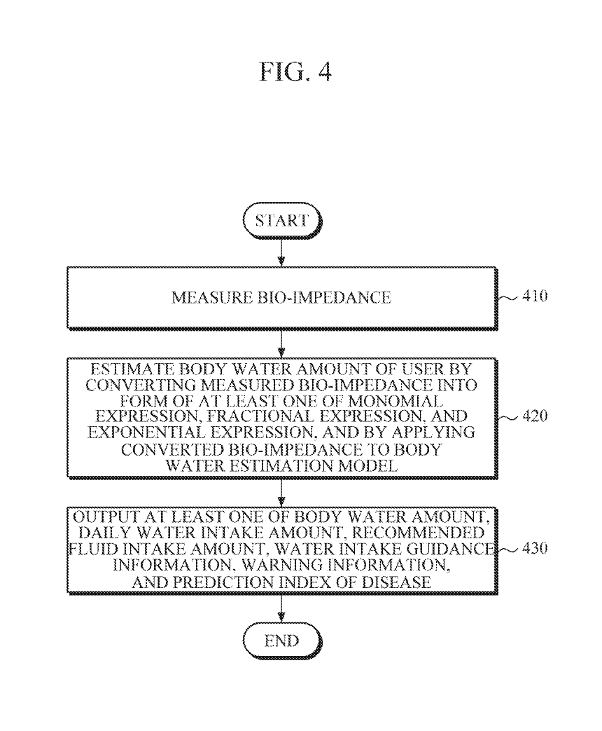

FIG. 4 is a flowchart illustrating an example of a bio-information processing method. The bio-information processing method may be performed by the bio-information processing apparatuses 100, 200, and 300 illustrated in FIGS. 1, 2, and 3A to 3E.

The bio-information processing apparatus 100 may measure bio-impedance Z in operation 410.

For example, the bio-information processing apparatus 100 may apply an alternating current between electrodes, which are in contact with the body, by using a two-electrode method or a four-electrode method, and may measure the bio-impedance by measuring voltage between the electrodes.

Upon measuring the bio-impedance, the bio-information processing apparatus 100 may estimate a body water amount of a user based on the measured bio-impedance.

For example, the bio-information processing apparatus 100 may estimate a body water amount of a user by converting the measured bio-impedance into the form of at least one of a monomial expression, a fractional expression, and an exponential expression, and by applying the converted bio-impedance to a body water estimation model in operation 420. For example, the bio-information processing apparatus 100 may convert the measured bio-impedance of the user based on the user's stature. In particular, the value of the user's stature may be raised to the power of n to obtain the value of statureⁿ, wherein n denotes a positive integer. In turn, the value of statureⁿ may be divided by the measured bio-impedance Z to obtain the value of statureⁿ/Z The obtained value statureⁿ/Z may be applied to the body water estimation model. However, the operation of the bio-information processing apparatus 100 is not limited thereto, and in the case where the constant applied in converting the bio-impedance is 1, the bio-information processing apparatus 100 may apply the measured bio-impedance Z as it is to the body water estimation model, in which case the bio-information processing apparatus 100 may omit the process of converting the measured bio-impedance.

Further, the bio-information processing apparatus 100 may apply a weighted value to at least one of the user information and the measured bio-impedance, and may estimate the body water amount by applying a result, obtained by applying the weighted value, to the body water estimation model.

For example, the body water amount is generally proportional to the stature and weight, and is inversely proportional to age, and men usually have a higher percentage of body water than women. Accordingly, the bio-information processing apparatus 100 may apply a weighted value to at least one of the user information and the measured bio-impedance, and may estimate an individualized body water amount by applying a result, obtained by applying the weighted value, to the body water estimation model.

The bio-information processing apparatus 100 may output at least one of a body water amount, a daily water intake amount, a recommended fluid intake amount, water intake guidance information, warning information, and the prediction index of diseases in operation 430.

For example, the bio-information processing apparatus 100 may generate guidance information or warning information based on a measurement result of the body water amount according to at least one usage mode among a normal mode, an exercise mode, a patient mode, and a user-defined mode.

For example, in the case where the usage mode is the normal mode, the bio-information processing apparatus 100 may generate water intake guidance information based on the input user information and the estimated body water amount. For example, the bio-information processing apparatus 100 may compare a reference body water amount with the estimated body water amount, and if the body water amount of a user is less than the reference body water amount by a predetermined amount (e.g., 2% less than the reference body water amount), the bio-information processing apparatus 100 may generate body water intake guidance information to induce a user to intake water.

In another example, in the case where the usage mode is the exercise mode, the bio-information processing apparatus 100 may continuously estimate a change in the estimated body water amount, and in the case where the change in the estimated body water amount exceeds a predetermined threshold value, the bio-information processing apparatus 100 may generate warning information. For example, while a user does an exercise or an activity which requires physically intense movements, loss of body water may result in a reduced exercise performance. In this case, if sufficient water is not supplied such that a pituitary gland may not maintain homeostasis of body water, problems may occur in regulation of body temperature or in the cardiovascular system.

Accordingly, if the usage mode is the exercise mode, the bio-information processing apparatus 100 may measure bio-impedance of a user according to a predetermined period (e.g., ten times/hour), and may estimate a body water amount based on the measured bio-impedance, in which the bio-information processing apparatus 100 may continuously estimate a change between the initially estimated body water amount or the body water amount estimated immediately before the current estimation, and the currently estimated body water amount. In this case, if the currently estimated body water amount is less than the initially estimated body water amount by a predetermined amount (e.g., 2% less than the initially estimated body water amount), or in the case where the currently estimated body water amount is less than the body water amount estimated immediately before the current estimation by a predetermined amount (e.g., 0.4% less than the body water amount estimated immediately before the current estimation), the bio-information processing apparatus 100 may generate warning information to warn of a sharp decrease in the body water amount.

As described above, the bio-information processing apparatus 100 may continuously estimate the body water amount based on the bio-impedance, and may calculate a change in the body water amount, such that even in the case where the body water amount is changed during a short period of time due to an intense activity such as an exercise, the bio-information processing apparatus 100 may generate warning information to maintain exercise performance of a user.

In yet another example, if the usage mode is the patient mode, the bio-information processing apparatus 100 may generate a prediction index of diseases including liver cirrhosis, intercapillary glomerulosclerosis, and edema, and prognosis evaluation information of a surgical operation field based on the input user information and the estimated body water amount.

For example, the human body water may be divided into intracellular water and extracellular water, a ratio of which is maintained at 3:2. In this case, the bio-information processing apparatus 100 may calculate a prediction index of diseases, including liver cirrhosis, intercapillary glomerulosclerosis, and edema, according to an extent that the estimated ratio of the intracellular water and the extracellular water falls outside a predetermined reference ratio (e.g., intracellular water:extracellular water=3:2).

Based on the calculated prediction index of diseases and prognosis evaluation information of the surgical operation filed, the bio-information processing apparatus 100 may generate guidance information regarding water intake or change of a resting posture, or may transmit an alarm (e.g., text message, warning information, etc.) to a related institution (e.g., specialized hospital, sanatorium, nursing care organization, etc.).

Further, the bio-information processing apparatus 100 may generate an alarm for periodically, constantly, or continuously measure the body water amount based on a user movement state and/or a measurement state of impedance which is measured by the impedance measurer 110.

For example, in the case where the bio-information processing apparatus 100 is implemented as a wearable device which may be worn on the wrist, impedance may not be stably measured by the impedance measurer 110 due to contact failure or motion noise caused while a user moves.

For example, the bio-information processing apparatus 100 may detect whether a user takes a break or does an activity based on the motion of the bio-information processing apparatus 100 which is sensed by a motion sensor (e.g., acceleration sensor, gyro sensor, proximity sensor, image sensor, etc.), and may generate an alarm for measuring the body water amount by determining a current activity state of a user.

For example, in the case where a user movement is detected by the motion sensor, the measured impedance Z may include many motion noises, or the measured impedance may be inaccurate due to incomplete contact of electrodes.

In this case, the bio-information processing apparatus 100 may determine whether a user takes a break by monitoring a user's movement using the motion sensor.

For example, in the case where no user movement is detected, the bio-information processing apparatus 100 may determine that a user takes a break, and may generate an alarm to induce measurement of the body water amount.

As described above, while a user is in a stable condition, the bio-information processing apparatus 100 induces measurement of the body water amount, such that the bio-information processing apparatus 100 may accurately measure the body water amount and may constantly manage the body water amount.

However, even in the case where a user movement is detected by the motion sensor, if a voltage, detected on two or four electrodes of the impedance measurer 110, is maintained in a normal range, or a current and a voltage are constantly detected on the two or four electrodes, such that the bio-information processing apparatus 100 determines that the electrodes normally contact an object, the bio-information processing apparatus 100 may continuously measure the body water amount.

For example, even when a user movement is detected, if a current and a voltage are constantly detected by each of the two or four electrodes, or the detected current and voltage are maintained in a normal range, the bio-information processing apparatus 100 may constantly estimate the body water amount by continuously measuring bio-impedance.

In addition, in the case where the bio-information processing apparatus 100, as a mobile device, is a wearable device which may be worn on the wrist, a motion caused in a user's daily life may constantly occur. In this case, the bio-information processing apparatus 100 may monitor motion information regardless of a usage mode (e.g., normal mode, exercise mode, patient mode, user-defined mode, etc.), and may generate a notification to induce measurement of the body water amount when the motion is stopped, or may automatically measure a user's body water amount.

In this manner, by measuring the body water amount based on a user movement state and/or an impedance measurement state, the bio-information processing apparatus 100 may continuously/periodically measure the body water amount even in constant motion.

The bio-information processing apparatus 100 may output at least one or more of an estimation result of body water, the generated guidance information and warning information according to a user's request or a predetermined period.

While not restricted thereto, an exemplary embodiment can be embodied as computer-readable code on a computer-readable recording medium. The computer-readable recording medium is any data storage device that can store data that can be thereafter read by a computer system. Examples of the computer-readable recording medium include read-only memory (ROM), random-access memory (RAM), CD-ROMs, magnetic tapes, floppy disks, and optical data storage devices. The computer-readable recording medium can also be distributed over network-coupled computer systems so that the computer-readable code is stored and executed in a distributed fashion. Also, an exemplary embodiment may be written as a computer program transmitted over a computer-readable transmission medium, such as a carrier wave, and received and implemented in general-use or special-purpose digital computers that execute the programs. Moreover, it is understood that in exemplary embodiments, one or more units of the above-described apparatuses and devices can include circuitry, a processor, a microprocessor, etc., and may execute a computer program stored in a computer-readable medium.

The foregoing exemplary embodiments are merely exemplary and are not to be construed as limiting. The present teaching can be readily applied to other types of apparatuses. Also, the description of the exemplary embodiments is intended to be illustrative, and not to limit the scope of the claims, and many alternatives, modifications, and variations will be apparent to those skilled in the art.

What is claimed is:

1. A bio-information processing apparatus comprising:
a main body;
a strap which is connected to both ends of the main body;
an impedance measurer configured to measure a bio-impedance of a user while the main body and the strap are in contact with the user; and
a processor configured to estimate a body water amount of the user by applying the measured bio-impedance to a body water estimation model.

2. The bio-information processing apparatus of claim 1, wherein the processor is further configured to convert the measured bio-impedance into at least one of a linear expression, a fractional expression, and an exponential expression, and estimate the body water amount of the user by applying the converted bio-impedance to the body water estimation model.

3. The bio-information processing apparatus of claim 1, further comprising an input interface configured to input at least one of user information and a usage mode,
wherein the user information comprises at least one of a gender of the user, an age of the user, a stature of the user, a weight of the user, a body mass index (BMI) of the user, and measurement position of the user.

4. The bio-information processing apparatus of claim 3, wherein the processor is further configured to apply a weighted value to at least one of the user information and the measured bio-impedance, and estimate the body water amount by applying a result, which is obtained by applying the weighted value to the at least one of the user information and the measured bio-impedance, to the body water estimation model.

5. The bio-information processing apparatus of claim 3, wherein the usage mode comprises a normal mode, an exercise mode, and a patient mode.

6. The bio-information processing apparatus of claim 5, wherein the processor is further configured to, in response to the usage mode being the normal mode, generate water intake guidance information based on the input user information and the estimated body water amount.

7. The bio-information processing apparatus of claim 5, wherein the processor is further configured to, in response to the usage mode being the exercise mode, continuously estimate a change in the estimated body water amount, and in response to the change in the estimated body water amount exceeding a predetermined threshold value, generate warning information.

8. The bio-information processing apparatus of claim 5, wherein the processor is further configured to, in response to the usage mode being the patient mode, generate at least one of a prediction index of diseases, which include liver cirrhosis, intercapillary glomerulosclerosis, and edema, and prognosis evaluation information of a surgical operation field based on the input user information and the estimated body water amount.

9. The bio-information processing apparatus of claim 1, wherein the processor is further configured to determine whether the user takes a break by monitoring movement of the user, and generate an alarm to induce measurement of the body water amount in response to determining that the user takes the break.

10. The bio-information processing apparatus of claim 1, wherein the impedance measurer comprises:
a first rear surface electrode and a second rear surface electrode which are disposed on a rear surface of the main body to directly come into contact with the user; and
a first front surface electrode and a second front surface electrode which are disposed on a front surface of the main body to come into contact with the user when body composition of the user is measured, and which are arranged to be touchable by a single finger of the user.

11. The bio-information processing apparatus of claim 10, wherein the impedance measurer is further configured to apply a current through the first rear surface electrode and the first front surface electrode, and measure the bio-impedance by measuring voltage between the second rear surface electrode and the second front surface electrode.

12. The bio-information processing apparatus of claim 10, wherein each of the first front surface electrode, the second front surface electrodes, the first rear surface electrode, and the second rear surface electrode is formed in at least one shape of a square, a circle, a concentric circle, and a semi-circle.

13. The bio-information processing apparatus of claim 1, further comprising an output interface configured to output at least one of a body water amount, a daily water intake amount, a recommended fluid intake amount, water intake guidance information, warning information, and a prediction index of diseases.

14. A bio-information processing method comprising:
measuring a bio-impedance; and
converting the measured bio-impedance into at least one of a linear expression, a fractional expression, and an exponential expression; and
estimating a body water amount of a user by applying the converted bio-impedance to a body water estimation model.

15. The bio-information processing method of claim 14, further comprising inputting at least one of user information and a usage mode,
wherein the user information comprises at least one of a gender of the user, an age of the user, a stature of the user, a weight of the user, a body mass index (BMI) of the user, and a measurement position of the user.

16. The bio-information processing method of claim 15, wherein the estimating the body water amount comprises:
applying a weighted value to at least one of the user information and the measured bio-impedance; and
estimating the body water amount by applying a result, which is obtained by applying the weighted value to the at least one of the user information and the measured bio-impedance, to the body water estimation model.

17. The bio-information processing method of claim 15, further comprising, in response to the usage mode being a normal mode, generating water intake guidance information based on the user information and the estimated body water amount.

18. The bio-information processing method of claim 15, further comprising, in response to the usage mode being an exercise mode, continuously estimating a change in the estimated body water amount, and in response to the change in the estimated body water amount exceeding a predetermined threshold value, generating warning information.

19. The bio-information processing method of claim 15, further comprising, in response to the usage mode being a patient mode, generating at least one of a prediction index of diseases, which include liver cirrhosis, intercapillary glomerulosclerosis, and edema, and prognosis evaluation information of a surgical operation field based on the user information and the estimated body water amount.

20. The bio-information processing method of claim 14, further comprising outputting at least one of a body water amount, a daily water intake amount, a recommended fluid intake amount, water intake guidance information, warning information, and a prediction index of diseases.
